(19) United States
(12) Patent Application Publication
(10) Pub. No.: US 2025/0235095 A1
(43) Pub. Date: Jul. 24, 2025

(54) SYSTEM FOR CONTROLLING AT LEAST ONE EYE EXAMINATION DEVICE

(71) Applicant: SIVIEW, PARIS (FR)

(72) Inventor: Nacer BOUSSAHOUL, BÈGLES (FR)

(21) Appl. No.: 18/853,625

(22) PCT Filed: Mar. 31, 2023
(86) PCT No.: PCT/EP2023/058485

§ 371 Date: Oct. 2, 2024

(30) Foreign Application Priority Data
Apr. 4, 2022 (FR) FR2203039

(51) Int. Cl. A61B 3/00; G16H 40/63

(52) U.S. Cl. CPC A61B 3/0025; A61B 3/0033; G16H 40/63

(57) ABSTRACT

A system for controlling at least one vision screening device configured to communicate according to a first communication protocol, includes: a control module configured to communicate according to a second communication protocol; for each device, a hardware key configured to be connected to the device and to the control module, and to ensure communication between the device and the control module through conversion of the first communication protocol and of the second communication protocol; the control module being configured to control each device, to receive data from each device via the corresponding hardware key and to process the received data.

### TECHNICAL FIELD OF THE INVENTION

The technical field of the invention is that of eye examinations, and more particularly that of systems for controlling apparatuses for the implementation of eye examinations.

The present invention relates to a control system for performing an eye examination, and more particularly to a control system for at least one eye examination apparatus. The present invention also relates to an eye examination system comprising the control system and a control method implemented by the control system.

### TECHNOLOGICAL BACKGROUND OF THE INVENTION

An eye examination includes several steps, each involving one or more apparatuses, including:
optionally but frequently:
   an autorefractometer for objective measurement of characteristics of the eye, and
   a lens meter for measuring optical characteristics of the patient's spectacle lenses, if they already have some,
   a computer including patient management software;
systematically, unless a pair of trial spectacles or a manual refractor head is used:
   an automatic refractor head in order to place lenses in front of a patient's eye or eyes and a control console for the automatic refractor head in order to drive placement of the lenses,
   as well as an optotype screen or projector in order to display the tests to be read.

These apparatuses operate in a complementary manner, i.e. data supplied by one will often be used by another. This is the case, for example, with the autorefractometer, which supplies data that are then used to drive the automatic refractor head. Their interoperability is therefore essential to the automated implementation of an eye examination.

Communication between devices, when it is possible, remains generally limited to devices from a same manufacturer, or even from a same manufacturer's series. Indeed, each type of eye examination apparatus and each model of type of apparatus has its own mode of operation, in particular:
in terms of connectors, there is a huge diversity of plug types, and a tendency for manufacturers not to comply with standards and/or practices. A plug normally used for computer screen video can thus be used to connect an automatic refractor head;
several different communication protocols, of the bus or point-to-point type, is likely to be used: RS232, RS485, CanBus, etc.

This situation has a number of drawbacks, including:
practitioners are obliged to obtain their equipment exclusively from one manufacturer, unless they wish to forego interoperability of their apparatus;
if an apparatus breaks down and it is impossible to replace it identically, for example because the manufacturer's range has changed, the need to also replace other apparatuses that are still functional;
the coexistence of varied user interfaces, specific to each manufacturer or even each model of apparatus, which requires protocols to be adapted and staff to be trained;
the impossibility of benefiting from diagnostic assistance tools, self- diagnostics or any other third-party improvements due to a closed proprietary design;
the hindrance to the emergence of competitive products that do not have a complete range available.

To overcome these drawbacks, it is known to resort to an interconnection box enabling communication between the different apparatuses connected to it, by converting between the different communication protocols used. The interconnection box is then driven via a user device on which a dedicated application has been installed for controlling the apparatuses via the interconnection box. For this, the interconnection box has a dedicated hardware port for each apparatus, for example a DE9, DA15 or DVI port, and a protocol management card. However, for a same apparatus, the connector type and the communication protocol used differ between the different models. Thus, the interconnect box will have to be changed as soon as an apparatus is replaced with an apparatus with different connectors or a different communication protocol, even though the interconnect box is functional.

There is therefore a need to enable interoperability between different apparatuses used during an eye examination without having to change functional equipment when an apparatus has to be replaced.

### SUMMARY OF THE INVENTION

The invention offers a solution to the problems previously discussed, by enabling automated implementation of an eye examination by ensuring communication between the different apparatuses used during the examination, without having to change the interconnection box when an apparatus is replaced with a different connector type or protocol apparatus.

A first aspect of the invention relates to a control system for a plurality of eye examination apparatuses, each apparatus being configured to communicate according to a first communication protocol specific to the apparatus, the control system comprising:
a control box configured to communicate according to a second communication protocol;
for each apparatus, a respective hardware key configured to be connected to the apparatus and to the control box, and to ensure communication between the apparatus and the control box by converting the first communication protocol and the second communication protocol;
   the control box comprising a plurality of hardware ports, each hardware key being configured to be connected to the control box via a respective hardware port of the control box;
   the control box being configured to control each apparatus via the corresponding hardware key, to receive data from each apparatus via the corresponding hardware key and to process the data received.

By virtue of the invention, performing the eye examination is automatically managed by the control box which, for each step of the eye examination, sends commands to each eye examination apparatus involved in the performance of the step, and which recovers and processes the data sent by each apparatus during or at the end of this performance, in order to prepare the commands to be sent to the apparatuses performing the following steps, or in order to prepare the final report.

Each eye examination apparatus is connected to the control box via a hardware key, which means that the control box does not have to be changed as soon as one model of eye examination apparatus is replaced with another model with different connector type. It will then be sufficient to change the hardware key.

In addition, each hardware key ensures communication between the eye examination apparatus connected to it and the control box by managing communication protocols, which means that the control box does not have to be changed as soon as one model of eye examination apparatus is replaced with another model using a different communication protocol.

In particular, each hardware key provides conversion between the physical communication protocols (i.e. the physical layer protocols) of the control box on the one hand, and of the apparatus to which it is attached on the other hand.

For its part, the control box translates the software communication protocols (i.e. the machine languages) of the control instructions it receives from an external user device (e.g. a tablet).

This makes it possible to obtain a system comprising a box having an input interface for receiving commands for eye examination apparatuses and a plurality of outputs for attaching hardware keys for connecting the box to the apparatus. The outputs of the control box can advantageously be outputs with standard connectors, for example USB type connectors, which avoids having to design a box incorporating connectors specifically adapted to some apparatuses, which may no longer be adapted if the apparatuses are changed. Each hardware key can thus comprise a standard port to be attached to the box, and a specific port (in accordance with the device protocol) to be attached to the corresponding apparatus. Thereby, each hardware key converts physical protocols between the box and the apparatus to which it is attached. Such a system therefore allows greater flexibility, and especially makes it easy to adapt in the event of new apparatuses being added or apparatuses being replaced with apparatuses operating according to protocols other than the previous apparatuses.

Further to the characteristics just discussed in the previous paragraph, the method according to the invention may have one or more additional characteristics from among the following ones, considered individually or according to any technically possible combinations.

According to one alternative embodiment, each hardware key includes a first hardware port adapted to the connector type of the corresponding apparatus.

According to one alternative embodiment compatible with the previous alternative embodiment, the control box is further configured to receive instructions corresponding to a control of an apparatus.

Thus, the practitioner can drive the control box, which prepares and sends commands to the eye examination apparatus as a function of the instructions they have received.

According to a first sub-alternative to the previous alternative embodiment, the control system according to the invention further comprises user equipment including a human computer interface, the instructions being input via the human computer interface and the user equipment being configured to transfer the instructions input to the control box.

Thus, the user equipment constitutes a single control panel for all the eye examining apparatuses connected to the control box.

According to a second sub-alternative embodiment to the previous alternative embodiment compatible with the first sub-alternative embodiment, the user equipment is connected to the control box via a wireless connection.

Thus, the control box can be remotely driven, which avoids the need for the practitioner to be on site to carry out the eye examination automatically implemented.

According to a third sub-alternative embodiment to the previous alternative embodiment compatible with the first and second sub-alternative embodiments, a hardware key is configured to be further connected to a control device, said control device being configured to control one apparatus among the plurality of apparatuses.

In one or more embodiments, the instructions received are instructions corresponding to a control of the apparatus controlled by the control device, said instructions comprising an mode of operation of the control box selected from a control mode and a supervision mode. The control box may be configured to: block communication between the apparatus and the control device via the corresponding hardware key, control the apparatus via the corresponding hardware key, receive data from the apparatus via the corresponding hardware key and process the data received when the instructions comprise the control mode; or unblock communication between the apparatus and the control device via the corresponding hardware key when the instructions comprise the supervision mode.

Thus, the practitioner can choose to carry out a test manually using the control device of the apparatus enabling the test to be carried out without having to disconnect the control box. The control box is then in supervision mode.

According to one alternative embodiment compatible with the previous alternative embodiments, the hardware key includes a second hardware port adapted to the connector type of the corresponding control device.

According to one alternative embodiment compatible with the previous alternative embodiments, the control box is further configured to receive and process data exchanged between the apparatus and the control device via the corresponding hardware key when the instructions comprise the supervision mode.

Data exchanged may be, for example, data relating to the eye examination (for example, examination results). This information may subsequently be used to control other apparatuses (for example, the data may comprise information relating to an evaluation of a refractive error of the eye obtained using an autorefractometer, and these data may be sent to an automatic refractor head). Thus, the control box in supervision mode collects data exchanged during the manual test carried out using the apparatus control device so that the following tests can be carried out automatically.

Data exchanged may also comprise operating data relating to the apparatus and/or the control device. For example, these data may comprise information relating to failures or abnormal operation of the apparatus and/or control device. Thus, it is possible to monitor state of health of the different apparatuses and devices of the system, and to plan maintenance interventions or updates if necessary.

Data exchanged may comprise other information, for example time-stamping information such as the date and time at which commands are sent from the control device to the apparatus, or data relating to a bandwidth on the communication link between the apparatus and the control device.

A second aspect of the invention relates to an eye examination system comprising the control system according to the invention and a plurality of eye examination apparatuses, each apparatus being connected to a respective hardware key of the control system.

Thus, one aspect of the invention relates to an eye examination system comprising:
a plurality of eye examination apparatuses, each apparatus being configured to communicate according to a first communication protocol specific to the apparatus;
a control box configured to communicate according to a second communication protocol;
for each apparatus, a respective hardware key configured to be connected to the apparatus and to the control box, and to ensure communication between the apparatus and the control box by converting the first communication protocol and the second communication protocol;
   the control box comprising a plurality of hardware ports, each hardware key being configured to be connected to the control box via a respective hardware port of the control box.

According to one alternative embodiment, the control system according to the invention further comprises, for an apparatus, a control device configured to control said apparatus, the control device being connected to the hardware key of the control system to which the corresponding apparatus is connected.

A third aspect of the invention relates to a method for controlling at least one eye examination apparatus implemented by the control system according to the invention, including the following steps of:
Receiving by the control unit instructions corresponding to a control of an apparatus;
If a first condition according to which the hardware key connected to the apparatus is not connected to a control device is met:
   Sending, by the control box, at least one command to the apparatus via the corresponding hardware key;
   Receiving, by the hardware key, the command sent;
   Converting, by the hardware key, the command received into the first communication protocol specific to the apparatus;
   Sending, by the hardware key, the command converted to the apparatus;
   Receiving, by the hardware key, at least one piece of data sent by the apparatus in response to sending the command converted;
   Converting, by the hardware key, the piece of data received into the second communication protocol of the control box;
   Sending, by the hardware key, the piece of data converted to the control unit;
   Receiving and processing, by the control box, the piece of data converted;
If the first condition is not met:
   If a second condition is met, according to which the instructions received comprise the control mode:
      Blocking, by the control unit, communication between the apparatus and the control device via the corresponding hardware key;
      Performing the steps if the first condition is met;
   If the second condition is not met:
      Unblocking, by the control box, communication between the apparatus and the control device via the corresponding hardware key.

According to one alternative embodiment, the method according to the invention further comprises the following steps performed following the unblocking step:
I Receiving, by the hardware key, at least one piece of data exchanged between the apparatus and the control device;
Converting, by the hardware key, the piece of data received into the second communication protocol of the control box;
Sending, by the hardware key, the piece of data converted to the control box;
Receiving and processing, by the control unit, the piece of data converted.

The invention and its different applications will be better understood upon reading the following description and upon examining the accompanying figures.

### BRIEF DESCRIPTION OF THE FIGURES

The figures are set forth by way of indicating and in no way limiting purposes of the invention.

FIG. 1 shows a schematic representation of an eye examination system according to the invention, comprising a control system according to the invention.

FIG. 2 is a block diagram illustrating the sequence of steps in a method according to the invention.

### DETAILED DESCRIPTION

Unless otherwise specified, a same element appearing in different figures has a single reference.

A first aspect of the invention relates to a control system for carrying out an eye examination, and more particularly to a control system for at least one eye examination apparatus.

The eye examination apparatus is, for example, an autorefractometer, a lens meter, an automatic refractor head or an optotype screen or projector.

Each eye examination apparatus uses its own communication protocol, hereinafter referred to as the first communication protocol.

The first communication protocol is, for example, RS232, RS485 or CanBus.

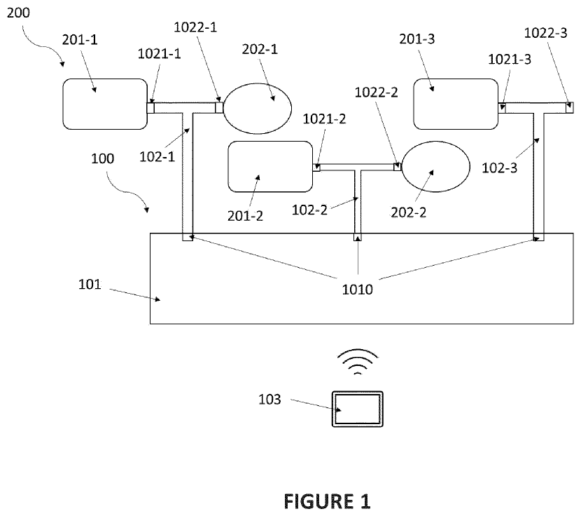

FIG. 1 shows a schematic representation of the control system 100 according to the invention.

The control system 100 comprises:
a control box 101;
a hardware key 102-1, 102-2, 102-3 for each eye examination apparatus 201-1, 201-2, 201-3.

Each hardware key 102-1, 102-2, 102-3 is configured to be connected to the corresponding apparatus 201-1, 201-2, 201-3. For this, each hardware key 102-1, 102-2, 102-3 includes, for example, a first hardware port 1021-1, 1021-2, 1021-3 adapted to the connector type of the corresponding apparatus 201-1, 201-2, 201-3.

The first hardware port 1021-1, 1021-2, 1021-3 is for example a DE9, DA15 or DVI port.

In FIG. 1, the control system 100 comprises a first hardware key 102-1 connected to a first eye examination apparatus 201-1, a second hardware key 102-2 connected to a second eye examination apparatus 201-2 and a third hardware key 102-3 connected to a third eye examination apparatus 201-3. The first hardware key 102-1 includes a first hardware port 1021-1, the second hardware key 102-2 includes a first hardware port 1021-2, and the third hardware key 102-3 includes a first hardware port 1021-3.

Each hardware key 102-1, 102-2, 102-3 is also configured to be connected to the control box 101. For this, each hardware key 102-1, 102-2, 102-3 includes, for example, a plug adapted to a hardware port 1010 of the control box 101, the control box 101 then including one hardware port 1010 per hardware key 102-1, 102-2, 102-3.

In FIG. 1, each hardware key 102-1, 102-2, 102-3 is connected to the control box 101 via a hardware port 1010, the control box 101 therefore including three hardware ports 1010.

All the hardware ports 1010 of the control box 101 are for example identical. Each hardware port 1010 of the control box 101 is, for example, a USB port.

Each hardware key 102-1, 102-2, 102-3 is configured to ensure communication between the apparatus 201-1, 201-2, 201-3 and the control box 101 to which the hardware key 102-1, 102-2, 102-3 is connected. For this, each hardware key 102-1, 102-2, 102-3 performs conversion between the first communication protocol used by the apparatus 201-1, 201-2, 201-3 and a second communication protocol used by the control box 101.

The control box 101 is configured to control each apparatus 201-1, 201-2, 201-3 via the corresponding hardware key 102-1, 102-2, 102-3, i.e. to transmit commands to the apparatus 201-1, 201-2, 201-3 via the corresponding hardware key 102-1, 102-2, 102-3.

The control box 101 is also configured to receive data from each apparatus 201-1, 201-2, 201-3 via the corresponding hardware key 102-1, 102-2, 102-3 and to process the data received.

Processing the piece of data received by an apparatus 201-1, 201-2, 201-3 makes it possible, for example, to prepare commands to be sent to another apparatus 201-1, 201-2, 201-3 or to prepare a final report comprising the results of the eye examination and a recommended prescription.

The control box 101 may also be configured to receive instructions corresponding to a control from an apparatus 201-1, 201-2, 201-3. For this, the control system 100 includes, for example, user equipment 103 provided with a human computer interface via which the instructions can be input, the user equipment 103 then being configured to transfer the instructions input to the control box 101.

As illustrated in FIG. 1, the user equipment 103 is for example a tablet having a touch screen for human computer interface.

The user equipment 103 may also be a computer or a smartphone.

The user equipment 103 is for example connected to the control box 101 via a wireless connection, such as a WiFi or Bluetooth connection.

Alternatively, the user equipment 103 could also be connected to the control box 101 via a wired connection.

Each hardware key 102-1, 102-2, 102-3 may also be configured to be further connected to a control device 202-1, 202-2 dedicated to controlling the apparatus 201-1, 201-2, 201-3 to which the hardware key 102-1, 102-2, 102-3 is connected. For this, each hardware key 102-1, 102-2, 102-3 includes, for example, a second hardware port 1022-1, 1022-2, 1022-3 adapted to the connector type of the corresponding control device 202-1, 202-2.

For example, when the eye examination apparatus 201-1, 201-2, 201-3 is an automatic refractor head, the control device 202-1, 202-2 is, for example, its control console.

In FIG. 1, the first hardware key 102-1 is further connected to a first control device 202-1, the second hardware key 102-2 is further connected to a second control device 202-2 and the third hardware key 102-3 is not connected to any control device.

The first hardware key 102-1 includes a second hardware port 1022-1, the second hardware key 102-2 includes a second hardware port 1022-2, and the third hardware key 102-3 includes a second hardware port 1022-3.

In the case where a hardware key 102-1, 102-2, 102-3 is connected both to an apparatus 201-1, 201-2, 201-3 and to its control device 202-1, 202-2, instructions corresponding to a control of the apparatus 201-1, 201-2, 201-3 include a mode of operation of the control box 101 selected from a control mode and a supervision mode.

When the instructions comprise the control mode, the control box 101 is configured to block communication between the apparatus 201-1, 201-2, 201-3 and the control device 202-1, 202-2 via the hardware key 102-1, 102-2, 102-3, to control the apparatus 201-1, 201-2, 201-3 via the corresponding hardware key 1021, 102-2, 102-3, to receive data from the apparatus 201-1, 201-2, 201-3 via the corresponding hardware key 102-1, 102-2, 102-3, and to process the data received.

When the instructions comprise the supervision mode, the control box 101 is configured to unblock communication between the apparatus 201-1, 201-2, 201-3 and the control device 202-1, 202-2 via the corresponding hardware key 102-1, 102-2, 102-3.

When the instructions comprise the supervision mode, the control box 101 may also be configured to receive data exchanged between the apparatus 201-1, 201-2, 201-3 and the control device 202-1, 202-2 via the corresponding hardware key 102-1, 102-2, 102-3 and to process the exchanged data received.

Processing the exchanged data makes it possible to monitor the process carried out by the control device 202-1, 202-2 to enable the control box 101 to be able to control the apparatus 201-1, 201-2, 201-3 subsequently using some of the data from the process.

A second aspect of the invention relates to an eye examination system 200 comprising the control system 100 according to the invention and at least one eye examination apparatus 201-1, 201-2, 201-3.

Each eye examination apparatus 201-1, 201-2, 201-3 is connected to a hardware key 102-1, 102-2, 102-3 of the control system 100.

The eye examination system 200 may also include, for at least one apparatus 201-1, 201-2, 201-3, a control device 202-1, 202-2 dedicated to controlling the apparatus 201-1, 201-2, 201-3.

Each control device 202-1, 202-2 is connected to the hardware key 102-1, 102-2, 102-3 of the control system 100 to which the corresponding apparatus 201-1, 201-2, 201-3 is connected.

A third aspect of the invention relates to a control method for performing an eye examination, implemented by the control system 100 according to the invention.

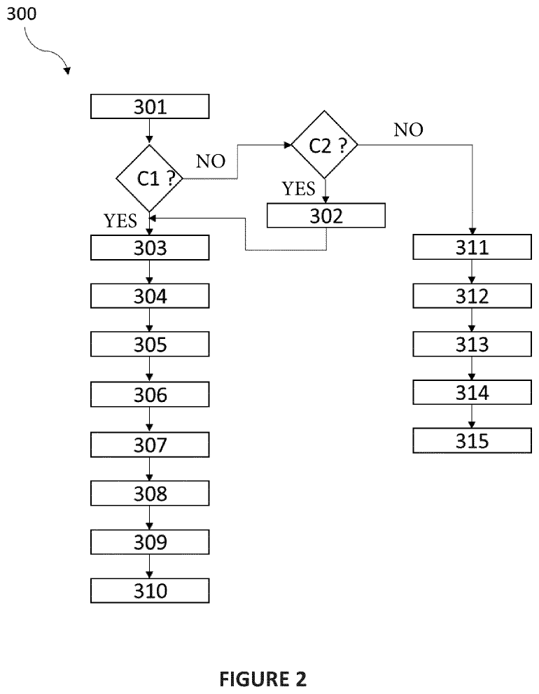

FIG. 2 is a synoptic diagram illustrating the sequence of steps of the control method 300 according to the invention.

A first step 301 of the control method 300 consists for the control box 101 in receiving instructions corresponding to the control of an eye examination apparatus 201-1, 201-2, 201-3.

If a first condition CI is met, third, fourth, fifth, sixth, seventh, eighth, ninth and tenth steps are performed.

The first condition CI is fulfilled if the hardware key 102-1, 102-2, 102-3 connected to the apparatus 201-1, 201-2, 201-3 to be controlled according to the instructions received in the first step 301, is not connected to a control device 202-1, 202-2.

The third step 303 of the control method 300 consists, for the control box 101, in sending at least one command to the apparatus 201-1, 201-2, 201-3 to be controlled, via the corresponding hardware key 102-1, 102-2, 102-3.

The fourth step 304 of the control method 300 consists, for the hardware key 102-1, 102-2, 102-3, in receiving the command sent by the control box 101 in the third step 303.

The fifth step 305 of the control method 300 consists, for the hardware key 102-1, 102-2, 102-3, in converting the command received in the fourth step 304 into the first communication protocol specific to the apparatus 201-1, 201-2, 2013 to be controlled.

The sixth step 306 of the control method 300 consists, for the hardware key 102-1, 102-2, 102-3, in transmitting the command converted in the fifth step 305 to the apparatus 201-1, 201-2, 201-3 to be controlled.

The seventh step 307 of the control method 300 consists, for the hardware key 102-1, 102-2, 102-3, in receiving at least one piece of data sent by the apparatus 201-1, 201-2, 201-3 to be controlled in response to sending the command converted.

The piece of data is, for example, relating to the view examination step implemented by the apparatus 201-1, 201-2, 201-3.

If the apparatus 201-1, 201-2, 201-3 to be controlled is a lens meter, the piece of data is, for example, an optical characteristic of the spectacle lenses of the patient for whom the eye examination is being carried out.

The eighth step 308 of the control method 300 consists, for the hardware key 102-1, 102-2, 102-3, in converting the piece of data received at the seventh step 307 into the second communication protocol of the control box 101.

The ninth step 309 of the control method 300 consists, for the hardware key 102-1, 102-2, 102-3, in sending the piece of data converted in the eighth step 308 to the control box 101.

The tenth step 310 of the control method 300 consists, for the control box 101, in receiving the piece of data converted sent in the ninth step 309 and in processing the piece of converted piece of data received.

If the first condition CI is not met, i.e. if the hardware key 102-1, 102-2, 102-3 connected to the apparatus 201-1, 201-2, 201-3, 301 to be controlled is connected to a control device 202-1, 202-2, and if a second condition C2 is met, a second step 302 is performed followed by the third, fourth, fifth, sixth, seventh, eighth, ninth and tenth steps.

The second condition C2 is met if the instructions received comprise the control mode.

The second step 302 of the control method 300 consists, for the control box 101, in blocking communication between the apparatus 201-1, 201-2, 201-3 and the control device 202-1, 202-2 via the corresponding hardware key 102-1, 102-2, 102-3.

If the second condition C2 is met, i.e. if the instructions comprise the supervision mode, an eleventh step is performed.

The eleventh step 311 of the control method 300 consists, for the control box 101, in unblocking communication between the apparatus 201-1, 201-2, 201-3 and the control device 202-1, 202-2 via the corresponding hardware key 102-1, 102-2, 102-3.

If the second condition C2 is met, twelfth, thirteenth, fourteenth and fifteenth steps may also be performed following the eleventh step 311.

The twelfth step 312 of the control method 300 consists, for the hardware key 102-1, 102-2, 102-3, in receiving at least one piece of data exchanged between the apparatus 201-1, 201-2, 201-3 and the control device 202-1, 202-2.

The thirteenth step 312 of the control method 300 consists, for the hardware key 102-1, 102-2, 102-3, in converting the piece of data received in the twelfth step 312 into the second communication protocol of the control box 101.

The fourteenth step 314 of the control method 300 consists, for the hardware key 102-1, 102-2, 102-3, in sending the piece of data converted in the thirteenth step 313 to the control box 101.

The fifteenth step 315 of the control method 300 consists, for the control box, in receiving the piece of data converted sent in the fourteenth step 314 and in processing the piece of data received.

It is noted that, in some embodiments, no eye examination apparatus 201-1, 201-2, 201-3 is connected to a control device 202-1, 202-2. According to these embodiments, the hardware keys 102-1, 102-2, 102-3 of FIG. 1 each comprise a plug adapted to a hardware port 1010 of the control box 101 (and thus allowing connection between the hardware key and the control box) and a hardware port 1021-1, 1021-2, 1021-3 adapted to the connector type of the corresponding apparatus 201-1, 201-2, 201-3 (and thus allowing connection between the hardware key and the corresponding apparatus).

In these embodiments where no eye examination apparatus 201-1, 201-2, 201-3 is connected to a control device 202-1, 202-2, the control method described above with reference to FIG. 2 may be simplified. Test C1 may be omitted, and steps 303 to 310 may be implemented in response to step 301 of receiving instructions corresponding to the control of an eye examination apparatus 201-1, 201-2, 201-3.

1. A control system for controlling a plurality of eye examination apparatuses, each apparatus being configured to communicate according to a first communication protocol specific to the apparatus, the control system comprising:
a control box configured to communicate according to a second communication protocol;
for each apparatus, a respective hardware key configured to be connected to the apparatus and to the control box, and to ensure communication between the apparatus and the control box by converting the first communication protocol and the second communication protocol;
the control box comprising a plurality of hardware ports, each hardware key being configured to be connected to the control box via a respective hardware port of the control box;
the control box being configured to control each apparatus via the corresponding hardware key, to receive data from each apparatus via the corresponding hardware key and to process the data received.

2. The control system according to claim 1, wherein each hardware key includes a first hardware port adapted to the connector type of the corresponding apparatus.

3. The control system according to claim 1, wherein the control box is further configured to receive instructions corresponding to a control of an apparatus.

4. The control system according to claim 3, further comprising user equipment including a human computer interface, the instructions being input via the human computer interface and the user equipment being configured to transfer the instructions input to the control box.

5. The control system according to claim 1, wherein a hardware key is configured to be further connected to a control device, said control device being configured to control one apparatus of the plurality of apparatuses.

6. The control system according to claim 5, wherein the control box is further configured to receive instructions corresponding to a control of an apparatus and wherein the instructions received are instructions corresponding to a control of the apparatus controlled by the control device, said instructions comprising a mode of operation of the control box selected from a control mode and a supervision mode; wherein the control box is configured to:
block communication between the apparatus and the control device via the corresponding hardware key, control the apparatus via the corresponding hardware key, receive data from the apparatus via the corresponding hardware key and process the data received when the instructions comprise the control mode; or
unblock communication between the apparatus and the control device via the corresponding hardware key when the instructions comprise the supervision mode.

7. The control system according to claim 5, wherein the hardware key includes a second hardware port adapted to the connector type of the corresponding control device.

8. The control system according to claim 6, wherein the control box is further configured to receive and process data exchanged between the apparatus and the control device via the corresponding hardware key, when the instructions comprise the supervision mode.

9. An eye examination system comprising the control system according to claim 1 and a plurality of eye examination apparatuses, each apparatus being connected to a respective hardware key of the control system.

10. The eye examination system according to the preceding claim 9, further comprising, for an apparatus, a control device configured to control said apparatus, the control device being connected to the hardware key of the control system to which the corresponding apparatus is connected.

11. A method for controlling at least one eye examination apparatus implemented by the control system according to claim 1 the method comprising:
receiving, by the control box, instructions corresponding to a control of an apparatus;
if a first condition according to which the hardware key connected to the apparatus is not connected to a control device is met:
sending, by the control box, at least one command to the apparatus to the corresponding hardware key;
receiving, by the hardware key, the command sent;
converting, by the hardware key, the command received into the first communication protocol specific to the apparatus;
sending, by the hardware key, the command converted to the apparatus;
Receiving receiving, by the hardware key, at least one piece of data sent by the apparatus in response to sending the command converted;
converting, by the hardware key, the piece of data received in the second communication protocol of the control box;
sending, by the hardware key, the piece of data converted to the control box;
receiving and processing, by the control unit, the piece of data converted;
if the first condition is not met:
if a second condition according to which the instructions received comprise the control mode is met:
blocking, by the control box, communication between the apparatus and the control device via the corresponding hardware key;
performing the steps carried out if the first condition is met;
if the second condition is not met:
unblocking, by the control box, communication between the apparatus and the control device via the corresponding hardware key.

12. The control method according to claim 10, further comprising the following steps performed following the unblocking step:
receiving, by the hardware key, at least one piece of data exchanged between the apparatus and the control device;
converting, by the hardware key, the piece of data received in the second communication protocol of the control box;
sending, by the hardware key, the piece of data converted to the control box;
receiving and processing, by the control box, the piece of data converted.
